(19) United States
(12) Patent Application Publication
(10) Pub. No.: US 2024/0009374 A1
(43) Pub. Date: Jan. 11, 2024

(54) Connector for Introducing Ambient Air Into a Negative Pressure Wound Therapy System

(71) Applicant: DeRoyal Industries, Inc., Powell, TN (US)

(72) Inventors: Dhanvin S. Desai, Knoxville, TN (US); Vincent D. Jardret, Powell, TN (US); Gavin L. Warrington, Powell, TN (US)

(73) Assignee: DeRoyal Industries, Inc., Powell, TN (US)

(21) Appl. No.: 18/220,122

(22) Filed: Jul. 10, 2023

Related U.S. Application Data
(60) Provisional application No. 63/359,420, filed on Jul. 8, 2022.

(51) Int. Cl. A61M 1/00

(52) U.S. Cl. CPC A61M 1/912; A61M 1/94; A61M 1/96; A61M 1/964

(57) ABSTRACT

A connector for a NPWT system includes a first section and a second section. The first section has a first reduced pressure lumen and a first secondary pressure lumen for delivering reduced pressure from a pump unit to a primary wound site through the reduced pressure lumen and secondary pressure from the pump unit to the primary wound site through the first secondary pressure lumen. The second section has a second reduced pressure lumen and a vent lumen with the second reduced pressure lumen being in fluid communication with the first reduced pressure lumen of the first section for delivering reduced pressure from the pump unit to a secondary wound site through the second reduced pressure lumen and the vent lumen being in fluid communication with the atmosphere for delivering secondary pressure from the atmosphere to the secondary wound site through the vent lumen.

### CROSS REFERENCE TO RELATED APPLICATION

This application claims priority to co-pending U.S. Provisional Application Ser. No. 63/359,420 filed Jul. 8, 2022, entitled “Y-Connector for Introducing Ambient Air into a Negative Pressure Wound Therapy System,” the entire contents of which is incorporated herein by reference.

### FIELD

This disclosure relates to the field of negative pressure wound therapy. More particularly, this disclosure relates to lumen connectors for introducing filtered air into a negative pressure wound therapy system to help facilitate the flow of exudate through the system.

### BACKGROUND

The primary purpose of negative pressure wound therapy (also referred to as “reduced pressure therapy” or “vacuum therapy” in the medical community) is to apply a reduced pressure (also referred to as “negative pressure” or “vacuum pressure” but will be referred to herein as “reduced pressure”) to a wound site to stimulate healing and remove excess exudate from the wound site. The major components of a negative pressure wound therapy system (hereinafter “NPWT system”) include a reduced pressure source (e.g., vacuum pump), a waste canister for collecting extracted exudate from the wound site, a wound dressing system for covering the wound site, and tubing fluidly connecting the wound dressing system to the reduced pressure source and the canister. The wound dressing system typically includes a wound filler such as foam or gauze and a thin film (typically a polyurethane film) adhesively applied to a patient's skin around the wound site and over the wound filler. The thin film creates a substantially sealed enclosure at the wound site. A hole is provided in the thin film near the center of the wound for enabling the wound dressing to be fluidly coupled to the reduced pressure source via a connector (e.g., “dome” or “bridge”). The connector fluidly connects the reduced pressure tubing to the hole in the thin film while maintaining the sealed environment around the wound. For purpose of the present disclosure, “fluidly connects” and “fluidly connected” refers to two or more components or lumens being in fluid communication with each other whether connected directly to each other or via intervening components such as connectors, adapters, etc. On the other hand, two components being in “direct fluid communication” or “connected directly” refers to the fluid communication of the components being the result of the components being directly connected to each other without any intervening components.

To assist the reduced pressure source, it has been determined that the presence of an additional air flow at the wound site plays a significant role in moving the exudate from the wound site to the canister. For example, as described in detail in the present Applicant's U.S. Patent Publication No. 2019/0201595, the disclosure of which is incorporated by reference herein in its entirety, the NPWT system may further include a secondary pressure source configured to deliver a secondary pressure through secondary pressure tubing to the wound enclosure (i.e., along a second fluid flow path to the wound that is distinct from the first fluid flow path containing the reduced pressure). According to certain embodiments, the secondary pressure source may be an outlet of the same vacuum pump that is delivering the reduced pressure to the wound enclosure. In other embodiments, the secondary pressure source is a second pump or an air vent disposed in the pump unit. The secondary pressure includes a positive pressure as compared to the reduced pressure being delivered from the reduced pressure source such that the secondary pressure helps facilitate the removal of exudate through the reduced pressure tubing when the two flow paths converge at the wound site. In certain embodiments, at least a portion of the reduced pressure tubing containing the first fluid flow path and the secondary pressure tubing containing the second fluid flow path may be coupled to each other using dual lumen tubing as known in the art.

With reference to FIG. 1, Y-connectors are known to be used in NPWT systems to provide reduced pressure therapy to multiple wounds while using a single pump unit. As shown in the simplified schematic of FIG. 1, the NPWT system 10 includes a pump unit 12 fluidly connected to a proximal end 22 of the dual lumen tubing 20 (with a canister unit being fluidly connected to the pump unit as known in the art for receiving exudate removed from the wound site as known in the art). The dual lumen tubing 20 includes a reduced pressure lumen 24 and a secondary pressure lumen 26. Y-connector 30 is fluidly connected to the distal end 28 of the tubing 20. As shown, the Y-connector 30 of the prior art is used to deliver the reduced pressure from the pump unit 12 to both Wound 1 (primary wound site) and Wound 2 (secondary wound site).

More specifically, the connector 30 includes a first wing portion 32 having a reduced pressure lumen 34 and a secondary pressure lumen 36. When the connector 30 is fluidly connected to the dual lumen tubing 20, the reduced pressure lumen 34 of the first wing portion 32 of the connector 30 is fluidly connected to the reduced pressure lumen 24 of the tubing 20 and the secondary pressure lumen 36 of the first wing portion 32 of the connector 30 is fluidly connected to the secondary pressure lumen 26 of the tubing 20 to provide for the flow of both reduced pressure and secondary pressure to the primary Wound 1 using two distinct flow paths (the flow paths then converge at the wound site whether through the wound itself or through the connector/dome used at the wound site as known in the art). The connector 30 then further includes a second wing portion 42 having only a reduced pressure lumen 44 that is in fluid communication with the reduced pressure lumen 34 of the first wing portion 32 of connector such that the reduced pressure lumen 44 is also fluidly connected to the reduced pressure lumen 24 of the tubing 20. Thus, only reduced pressure is delivered to the secondary Wound 2 site. While this prior art system allows for control of the pressure and removal of exudate at the primary wound site using the secondary pressure source, the secondary wound sites are at risk of stalled fluid/exudate because the secondary pressure is not being introduced to the secondary sites.

### SUMMARY

The above and other needs are met by a connector for a negative pressure wound therapy system having a first section and a second section. The first section includes a first reduced pressure lumen and a secondary pressure lumen, and the connector is configured to be fluidly connected to tubing for delivering reduced pressure from a pump unit to a primary wound site through the reduced pressure lumen and secondary pressure from the pump unit to the primary wound site through the secondary pressure lumen. The second section includes a second reduced pressure lumen and a vent lumen, the second reduced pressure lumen being in fluid communication with the first reduced pressure lumen of the first section for delivering reduced pressure from the pump unit to a secondary wound site through the second reduced pressure lumen and the vent lumen being in fluid communication with an atmosphere surrounding the connector for delivering secondary pressure from the atmosphere to the secondary wound site through the vent lumen.

According to certain embodiments, the tubing is dual lumen tubing having a proximal end and a distal end with the proximal end for fluidly connecting the dual lumen tubing to the pump unit. The connector includes a base portion configured to be fluidly connected to the distal end of the dual lumen tubing.

According to certain embodiments, the connector includes a base portion, the first section of the connector is a first wing portion extending from the base portion in a first direction, and the second section of the connector is a second wing portion extending from the base portion in a second direction different from the first direction. According to some embodiments, the vent lumen has a proximal end disposed along a length of the second wing portion and a distal end disposed at a distal end of the second wing portion. According to other embodiments, the vent lumen has a proximal end disposed adjacent the base portion and a distal end disposed at a distal end of the second wing portion.

According to certain embodiments, the vent lumen is a filtered vent lumen configured to provide the secondary pressure from the atmosphere at a flow rate of about 5 mL/min to about 100 mL/min.

According to certain embodiments, the vent lumen is a filtered vent lumen configured to provide the secondary pressure from the atmosphere at a flow rate of about 5 mL/min to about 50 mL/min.

According to certain embodiments, the vent lumen is a filtered vent lumen configured to provide the secondary pressure from the atmosphere at a flow rate of about 10 mL/min to about 20 mL/min.

According to another embodiment of the disclosure, a negative pressure wound therapy system for delivering a source of reduced pressure from a pump unit to a plurality of wound sites is provided. The plurality of wound sites includes at least a primary wound site, a first secondary wound site, and a second secondary wound site. The system includes a first connector and a second connector each having a first section and a second section. The first section of the first connector includes a first reduced pressure lumen for maintaining a flow of reduced pressure from the pump unit along a first flow path and a secondary pressure lumen for maintaining a flow of secondary pressure from the pump unit along a second flow path. The second section of the first connector includes a second reduced pressure lumen and a vent lumen, the second reduced pressure lumen in fluid communication with the first reduced pressure lumen of the first section for delivering reduced pressure from the pump unit to the second secondary wound site, and the vent lumen in fluid communication with an atmosphere surrounding the first connector for delivering secondary pressure from the atmosphere surrounding the first connector to the second secondary wound site. The second connector is configured to be fluidly connected to the first section of the first connector. The first section of the second connector includes a first section having a first reduced pressure lumen in fluid communication with the first reduced pressure lumen of the first connector for delivering reduced pressure from the pump unit along the first flow path to the primary wound site and a secondary pressure lumen in fluid communication with the secondary pressure lumen of the first connector for delivering secondary pressure from the pump unit along the second flow path to the primary wound site. The second section of the second connector includes a second reduced pressure lumen and a vent lumen, the second reduced pressure lumen in fluid communication with the first reduced pressure lumen of the first section of the second connector for delivering reduced pressure from the pump unit to the first secondary wound site, and the vent lumen in fluid communication with an atmosphere surrounding the second connector for delivering secondary pressure from the atmosphere surrounding the second connector to the first secondary wound site.

According to certain embodiments, the first connector includes a base portion for fluidly connecting the first connector to a distal end of a first section of dual lumen tubing. In some embodiments, a distal end of the first section of the first connector is configured to be fluidly connected to a proximal end of a second section of dual lumen tubing, and the second connector includes a base portion for fluidly connecting the second connector to a distal end of the second section of dual lumen tubing.

According to certain embodiments, the first section of the first and second connector is a first wing portion extending in a first direction and the second section of the first and second connector is a second wing portion extending in a second direction different from the corresponding first direction. In some embodiments, the vent lumen of the first and second connector has a proximal end disposed along a length of the second wing portion of the corresponding first and second connector and a distal end disposed at a distal end of the second wing portion of the corresponding first and second connector.

According to certain embodiments, the vent lumen of the first and second connector is a filtered vent lumen configured to provide the secondary pressure from the atmosphere at a flow rate of about 5 mL/min to about 100 mL/min.

According to certain embodiments, the vent lumen of the first and second connector is a filtered vent lumen configured to provide the secondary pressure from the atmosphere at a flow rate of about 5 mL/min to about 50 mL/min.

According to certain embodiments, the vent lumen of the first and second connector is a filtered vent lumen configured to provide the secondary pressure from the atmosphere at a flow rate of about 10 mL/min to about 20 mL/min.

### BRIEF DESCRIPTION OF THE DRAWINGS

Other embodiments of the disclosure will become apparent by reference to the detailed description in conjunction with the figures, wherein elements may not be to scale so as to more clearly show the details, wherein like reference numbers indicate like elements throughout the several views, and wherein:

FIG. 1 depicts a simplified schematic of a NPWT system for delivering reduced pressure to two different wound sites/enclosures using a Y-connector of the prior art.

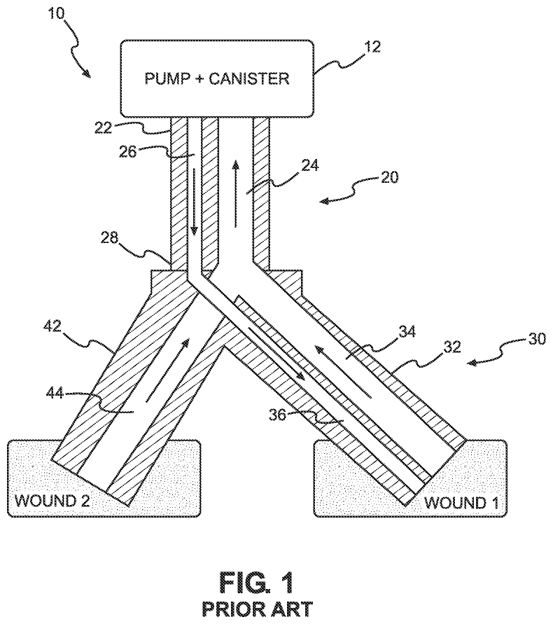

FIG. 2 depicts a simplified schematic of a NPWT system for delivering reduced pressure to two different wound sites/enclosures using a connector according to the present disclosure.

FIG. 3 depicts a simplified schematic of a NPWT system for delivering reduced pressure to a plurality of wound sites/enclosures using multiple connectors according to the present disclosure.

### DETAILED DESCRIPTION

The present disclosure is directed to lumen connectors for introducing ambient air to a NPWT system. The ambient air is introduced to help facilitate removal of fluid/exudate from a wound site to the NPWT canister. While the connector is shown and described herein as a “Y-connector,” other related configurations are possible and within the scope of the present disclosure (e.g., “T-shaped connector”).

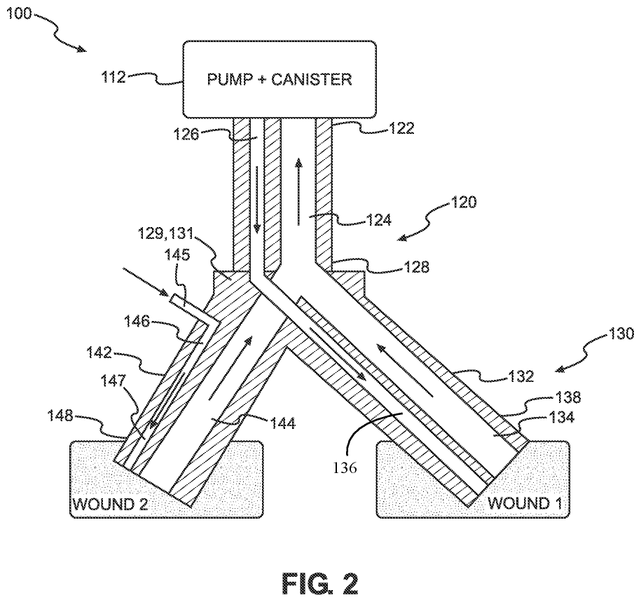

With reference to FIG. 2, the NPWT system 100 having connector 130 according to an exemplary embodiment of the present disclosure provides reduced pressure and secondary pressure to both a primary wound site (Wound 1) and a secondary wound site (Wound 2) using a single pump unit 112. According to this embodiment, the connector 130 includes proximal end 129 having a base portion 131, a first section 132 extending from the base portion 131 in a first direction, and a second section 142 extending from the base portion 131 in a second direction. The first section 132 is preferably a first wing portion projecting from the base portion 131 at a first angled direction, and the second section 142 is preferably a second wing portion projecting from the base portion 131 at a second angled direction away from the first angled direction. As shown, the base portion 131 is configured to fluidly connect to a distal end 128 of dual lumen tubing 120 while a proximal end 122 of the dual lumen tubing 120 is fluidly connected to the pump unit 112. Upon connection, the pump unit 112 is operable to (1) deliver both reduced pressure and secondary pressure through base portion 131 of connector 130 to a distal end 138 of the first section 132 such that reduced pressure and secondary pressure from the pump unit 112 is delivered to Wound 1; and (2) deliver reduced pressure through the base portion 131 of connector 130 to a distal end 148 of the second section 142 such that reduced pressure from the pump unit 112 is delivered to Wound 2. The second section 142 further then includes a filtered vent lumen 146 for introducing secondary pressure from the atmosphere to Wound 2.

More specifically, base portion 131 and first section 132 include a first reduced pressure lumen 134 and first secondary pressure lumen 136 that extends from the proximal end 129 of the connector 130 (i.e., end of connector 130 that is configured to be fluidly connected to the dual lumen tubing 120) to the distal end 138 of the first section 132 while keeping the first reduced pressure lumen 134 fluidly isolated from the first secondary pressure lumen 136 throughout the connector 130. When the base portion 131 of connector 130 is fluidly connected to the dual lumen tubing 120, the first reduced pressure lumen 134 of connector 130 is fluidly connected to the reduced pressure lumen 124 of dual lumen tubing 122 and the first secondary pressure lumen 136 of connector 130 is fluidly connected to the secondary pressure lumen 126 of dual lumen tubing 120. The distal end 138 of the first section 132 is then operable to be fluidly connected to the dressing system of Wound 1 to deliver both reduced pressure and secondary pressure from the pump unit 112 to Wound 1.

With continued reference to FIG. 2, the connector 130 further includes a second reduced pressure lumen 144 that is in fluid communication with the first reduced pressure lumen 134 within the base portion 131. The second reduced pressure lumen 144 extends from the fluid connection point with the first reduced pressure lumen 134 to a distal end 148 of second section 142 to provide reduced pressure from the pump unit 112 to Wound 2. To provide a source of secondary pressure to Wound 2, the connector 130 further includes a filtered vent lumen 146 disposed in the connector 130 and extending to the distal end 148 of the second section 142. The vent lumen 146 includes a proximal end 145 that is in fluid communication with the ambient environment outside of the connector 130 and a distal end 147 extending to the distal end 148 of second section 142 for delivering secondary pressure (i.e., filtered ambient air) to Wound 2 to facilitate removal of fluid/exudate from Wound 2 through second reduced pressure lumen 144.

In summary, connector 130 includes a first section 132 for maintaining the dual lumen flow from the pump unit 112 to the primary wound site (Wound 1) while the section 142 is configured to deliver reduced pressure from the pump unit 112 to a secondary wound site (Wound 2) while simultaneously providing secondary positive pressure from the atmosphere surrounding the connector 130 to Wound 2 to facilitate exudate removal from Wound 2.

In preferred embodiments, the proximal end 145 of the vent lumen 146 is disposed along the length of the second section 142/second wing portion. However, the proximal end 145 may also be disposed in the base portion 131 such that it then extends through the entirety of the second section 142 to Wound 2.

According to certain embodiments, the distal end 138 of first section 132 and/or distal end 148 of second section 142 are fluidly connected to corresponding Wound 1 and Wound 2 via a direct fluid connection to a wound site connector (e.g., dome connector). In other embodiments, the distal end 138 of first section 132 and/or distal end 148 of second section 142 may be configured to incorporate sufficient structure to be connected directly to corresponding Wound 1 or Wound 2. In the latter case, the connector 130 would typically be fluidly connected/sealed to Wound 1 and/or Wound 2 and then the dual lumen tubing 120 would be fluidly connected to the base portion 131 of connector 130. For purposes of the present disclosure, the connector 100 delivering reduced pressure or secondary pressure to a particular wound site shall be interpreted as delivering the pressure directly to the wound enclosure or to the wound enclosure via an intervening connector (e.g., dome connector).

In yet another potential embodiment, the distal end 138 of first section 132 and/or distal end 148 of second section 142 may be configured to receive an adapter for fluidly connecting the respective lumens to additional dual lumen tubing. In other words, either of the first and second sections 132 and 142 of connector 130 may be used to connect two different sections of dual lumen tubing such as described in U.S. Patent Publication 2020/0086017, which is commonly assigned to the present Applicant and the disclosure of which is incorporated by reference herein in its entirety. For example, the distal end 138 of first section 132 may be configured to be connected directly to a dome connector at Wound 1. However, if Wound 2 is disposed too far away from Wound 1, a dressing adapter such as described in U.S. Patent Publication No. 2020/0086017 may be provided to fluidly connect the distal end 148 of second section 142 to a second section of dual lumen tubing that then extends to Wound 2. In this case, vent lumen 146 of connector 130 still provides a source of secondary pressure that is delivered to Wound 2 via the secondary pressure lumen of the second section of dual lumen tubing.

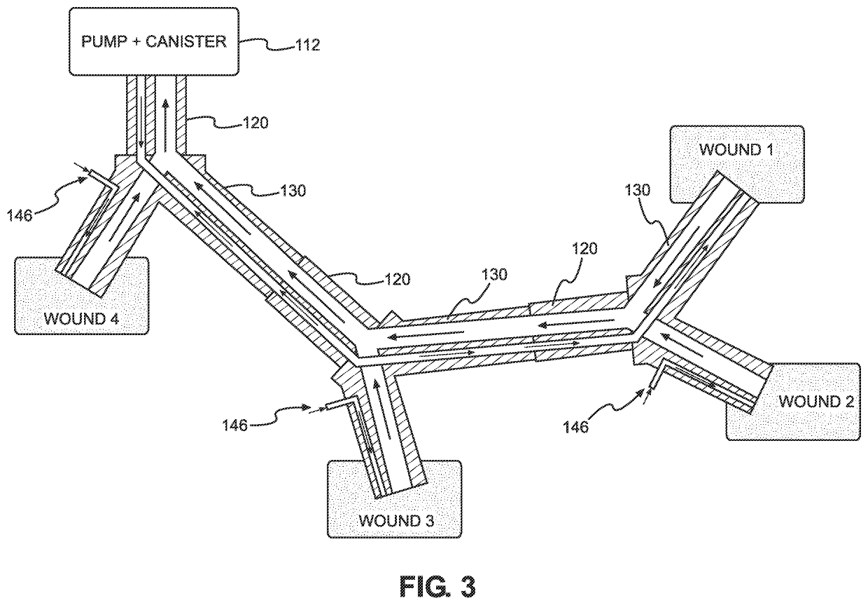

With reference to FIG. 3, and according to another embodiment of the disclosure, a plurality of connectors 130 may be daisy chained using additional sections of dual lumen tubing 120 to fluidly connect the system to multiple secondary wound sites (Wound 2, Wound 3, Wound 4). Each of connectors 130 provide for filtered ambient air to be introduced to all secondary wounds via the filtered vent lumens 146 of each connector 130.

In certain embodiments, the secondary pressure provided by the filtered vent lumen 146 of connector 130 is preferably in the range of about 5 mL/min to about 100 mL/min., more preferably in the range of about 5 mL/min to about 50 mL/min, and most preferably about 10 mL/min to about 20 mL/min. In embodiments with multiple connectors 130, air flow created by the vent lumens 146 provide secondary pressure to push the exudate toward the canister from all wounds being treated by the NPWT system.

According to another embodiment of the disclosure, a connector may be used for introducing filtered air into NPWT systems that do not have an existing secondary pressure source. For example, the pump unit may be used to provide only reduced pressure to a wound site using a tubing having a single reduced pressure lumen. When a source of secondary pressure is desired, connector is attached to the distal end of the tubing. According to this embodiment, the connector includes a reduced pressure lumen that is fluidly connected to the reduced pressure lumen of the tubing and a vent lumen that is fluidly connected to the reduced pressure lumen of the connector. The distal end of the connector could then be fluidly connected to the wound site.

According to another aspect of the disclosure, the connectors described herein may also be introduced at any point in the NPWT system to help move exudate along the reduced pressure flow path. For example, the connectors could be used to attach two different sections of tubing while introducing a source of positive pressure at the connection point. According to this example, the vent lumen of the connector would be in fluid communication with the reduced pressure lumen of the connector to provide a source of positive pressure at the point of the connector along the reduced pressure flow path.

According to another aspect of the disclosure, a valve may be provided for any of the vent lumens described herein such that secondary pressure is only provided to the NPWT system via the particular vent lumen when desired and/or needed.

The foregoing description of preferred embodiments for this disclosure have been presented for purposes of illustration and description. They are not intended to be exhaustive or to limit the disclosure to the precise form disclosed. Obvious modifications or variations are possible in light of the above teachings. The embodiments are chosen and described to provide the best illustrations of the principles of the disclosure and its practical application, and to thereby enable one of ordinary skill in the art to utilize the disclosure in various embodiments and with various modifications as are suited to the particular use contemplated. All such modifications and variations are within the scope of the disclosure as determined by any future claims when interpreted in accordance with the breadth to which they are fairly, legally, and equitably entitled.

What is claimed is:

1. A connector for a negative pressure wound therapy system, the connector comprising:
a first section having a first reduced pressure lumen and a secondary pressure lumen, the connector configured to be fluidly connected to tubing for delivering reduced pressure from a pump unit to a primary wound site through the reduced pressure lumen and secondary pressure from the pump unit to the primary wound site through the secondary pressure lumen; and
a second section having a second reduced pressure lumen and a vent lumen, the second reduced pressure lumen being in fluid communication with the first reduced pressure lumen of the first section for delivering reduced pressure from the pump unit to a secondary wound site through the second reduced pressure lumen and the vent lumen being in fluid communication with an atmosphere surrounding the connector for delivering secondary pressure from the atmosphere to the secondary wound site through the vent lumen.

2. The connector of claim 1 wherein the tubing is dual lumen tubing having a proximal end and a distal end, the proximal end for fluidly connecting the dual lumen tubing to the pump unit, and the connector including a base portion configured to be fluidly connected to the distal end of the dual lumen tubing.

3. The connector of claim 1 wherein the connector includes a base portion, the first section of the connector is a first wing portion extending from the base portion in a first direction, and the second section of the connector is a second wing portion extending from the base portion in a second direction different from the first direction.

4. The connector of claim 3 wherein the vent lumen has a proximal end disposed along a length of the second wing portion and a distal end disposed at a distal end of the second wing portion.

5. The connector of claim 3 wherein the vent lumen has a proximal end disposed adjacent the base portion and a distal end disposed at a distal end of the second wing portion.

6. The connector of claim 1 wherein the vent lumen is a filtered vent lumen configured to provide the secondary pressure from the atmosphere at a flow rate of about 5 mL/min to about 100 mL/min.

7. The connector of claim 1 wherein the vent lumen is a filtered vent lumen configured to provide the secondary pressure from the atmosphere at a flow rate of about 5 mL/min to about 50 mL/min.

8. The connector of claim 1 wherein the vent lumen is a filtered vent lumen configured to provide the secondary pressure from the atmosphere at a flow rate of about 10 mL/min to about 20 mL/min.

9. A negative pressure wound therapy system for delivering a source of reduced pressure from a pump unit to a plurality of wound sites including at least a primary wound site, a first secondary wound site, and a second secondary wound site, the system comprising:
a first connector including:
a first section having a first reduced pressure lumen for maintaining a flow of reduced pressure from the pump unit along a first flow path and a secondary pressure lumen for maintaining a flow of secondary pressure from the pump unit along a second flow path, and
a second section having a second reduced pressure lumen and a vent lumen, the second reduced pressure lumen in fluid communication with the first reduced pressure lumen of the first section for delivering reduced pressure from the pump unit to the second secondary wound site, and the vent lumen in fluid communication with an atmosphere surrounding the first connector for delivering secondary pressure from the atmosphere surrounding the first connector to the second secondary wound site; and
a second connector fluidly connected to the first section of the first connector, the second connector including:
a first section having a first reduced pressure lumen in fluid communication with the first reduced pressure lumen of the first connector for delivering reduced pressure from the pump unit along the first flow path to the primary wound site and a secondary pressure lumen in fluid communication with the secondary pressure lumen of the first connector for delivering secondary pressure from the pump unit along the second flow path to the primary wound site, and
a second section having a second reduced pressure lumen and a vent lumen, the second reduced pressure lumen in fluid communication with the first reduced pressure lumen of the first section of the second connector for delivering reduced pressure from the pump unit to the first secondary wound site, and the vent lumen in fluid communication with an atmosphere surrounding the second connector for delivering secondary pressure from the atmosphere surrounding the second connector to the first secondary wound site.

10. The system of claim 9 wherein the first connector includes a base portion for fluidly connecting the first connector to a distal end of a first section of dual lumen tubing.

11. The system of claim 10 wherein a distal end of the first section of the first connector is configured to be fluidly connected to a proximal end of a second section of dual lumen tubing, and the second connector includes a base portion for fluidly connecting the second connector to a distal end of the second section of dual lumen tubing.

12. The system of claim 9 wherein the first section of the first and second connector is a first wing portion extending in a first direction and the second section of the first and second connector is a second wing portion extending in a second direction different from the corresponding first direction.

13. The system of claim 12 wherein the vent lumen of the first and second connector has a proximal end disposed along a length of the second wing portion of the corresponding first and second connector and a distal end disposed at a distal end of the second wing portion of the corresponding first and second connector.

14. The system of claim 9 wherein the vent lumen of the first and second connector is a filtered vent lumen configured to provide the secondary pressure from the atmosphere at a flow rate of about 5 mL/min to about 100 mL/min.

15. The system of claim 9 wherein the vent lumen of the first and second connector is a filtered vent lumen configured to provide the secondary pressure from the atmosphere at a flow rate of about 5 mL/min to about 50 mL/min.

16. The system of claim 9 wherein the vent lumen of the first and second connector is a filtered vent lumen configured to provide the secondary pressure from the atmosphere at a flow rate of about 10 mL/min to about 20 mL/min.
